(19) United States
(12) Patent Application Publication
(10) Pub. No.: US 2015/0328444 A1
(43) Pub. Date: Nov. 19, 2015

(54) Capillary injection needle

(71) Applicant: Anton Blank, Stuttgart (DE)

(72) Inventor: Anton Blank, Stuttgart (DE)

(21) Appl. No.: 14/708,268

(22) Filed: May 10, 2015

(30) Foreign Application Priority Data
May 13, 2014 (DE) 102014006846.3

(51) Int. Cl. A61M 37/00

(52) U.S. Cl. CPC A61M 37/0015; A61M 2037/0023; A61M 2037/003; A61M 2037/0061

(57) ABSTRACT

Apparatus for the repeated injection of substances into an organic skin, with a housing (2) that is brought to reciprocating motion by a drive mechanism (1), and with at least one capillary injection needle (4) rigidly connected to the housing (2). The at least one capillary injection needle (4) is configured to transport a substance (6) by at least one capillary force through the at least one capillary injection needle (4) to its piercing tip (3), to at least partially moisten the piercing tip (3) with the substance (6), and to inject the substance (6) into an organic skin.

### TECHNICAL FIELD

The invention relates to a capillary injection needle for the injection of substances into an organic skin, and/or for the stimulation of an organic skin.

### BACKGROUND

Apparatuses for repeated injection into an organic skin are used e.g. for the decorative introduction of pigments for permanent make-up (PMU) or tattoos, or for the introduction of cosmetic or medical substances (mesotherapy). They may also be used for skin stimulation (regeneration provocation), using e.g. lubricants and/or substances having a therapeutic effects. The apparatuses generally consist of a drive unit (hand unit), which transmits a repetitive stroke motion to a piercing element that is capable of being removed from the hand unit and that is moistened with the substance to be introduced, in order to pierce the skin with each cycle of the stroke in order that the substance can penetrate into the skin and remain beneath it. The piercing may also be intended for the stimulation of the skin in order to provoke regeneration of the skin by creating tiny lesions.

The introduction of the substances or the piercing for the purpose of skin stimulation in a depth set by the user, i.e. a predetermined and precise depth in the skin, has thus far been neglected in practice, even though it is known in which optimum depths (skin layers) the various substances must be introduced depending on the chosen application, or in which skin depth effective stimulation must be carried out. Previously, it has been left to the user to manually set a piercing depth of only a few millimeters to a precision of less than a tenth of a millimeter, and to maintain this precise depth in the working motion of his hand along a distance of several centimeters or a surface of several square centimeters, which is impossible for the user, as the actual piercing depth is neither visible to nor measurable by the user during use.

EP2682146 discloses an apparatus with an adjustable piercing depth on a hand unit, the drive stroke depth of which is fixed and the needle and piercing depth setting device of which are moved synchronously by means of an actuator rod. The piercing depth is set by changing the distance of a forward end of a needle jet relative to the point of the needle (distal end), and—as already disclosed in EP000002671609—the front end of the needle jet functions as a limit stop on the skin, so that the needle can only penetrate into the skin to the depth that it projects beyond the end of the needle jet. This arrangement has the critical drawback that the zero point of the needle necessary in order to precisely set the piercing depth cannot be preset by either the manufacturer or the user, as all components are affected by certain tolerances as a result of the production process, and thus that the components to be swapped in and out for each application, which differ in length, meet a hand unit also differing in length. To illustrate the scale at issue here: the most common applications in practice require insertion depths of 0.05 mm to 0.3 mm in an organic skin layer (dermis). The calibration of the zero point of the needle tip relative to the end of the needle jet would have be significantly smaller than 0.05 mm (50 μm) to be sufficiently precise for practical use, and could only be performed directly by the user, but a value this small cannot be set by a human even with the aid of a magnifying glass.

While the piercing depth can be varied with this apparatus, an exactly defined depth in the necessary range of depths cannot be achieved with it, so it remains possible to work only at depths that are usually too great and are harmful for the client.

European Patent Applications EP000002671609 and EP000002671610 solve the tolerance problem by providing that a single component, namely the piercing tip, is used for determining the precise piercing depth. The manufacture of tip lengths even in the smallest required size range of 50 μm and a precision in setting the length of only a few μm can today be accomplished with ease.

### SUMMARY

The present disclosure provides an apparatus with improved injection characteristics of substances using capillary effects and the characteristics of the piercing tip for an application-related and substance-related piercing into an organic skin.

The apparatus provides one or more capillary injection needles that are hollow and allow unobstructed flow of a substance. Their piercing tips are either formed directly in the capillary material, or consist of separately produced piercing tips which are affixed later.

Known capillary injection devices provided a piercing element (needle) disposed within a capillary tube. Due to production tolerances the needle in such known devices were not perfectly centered, but subject to varying inclined position in the capillary which caused variation in the capillary forces. This disadvantage of the prior art is eliminated by the present invention. Without an internal needle, the capillary forces can now be calibrated more precisely for a specific application and manufacturing consistency can be maintained more easily during the production process.

An improved capillary injection needle comprises a piercing tip that is formed by machining the end of a capillary tube. Different tip shapes advantageous for specific applications are produced directly on the capillary. This results in an easier and more economical production of the capillary injection needles.

Alternatively, piercing tips may be manufactured separately and attached to capillary tubes to form a capillary injection needle. This allows manufacturing additional tip forms that are advantageous for specific applications and that could not be shaped as an integral part of a capillary tube. Such separately manufactured piercing tips are then affixed to the capillary tube in a separate production step.

The disclosed capillary injection needle may utilize an at least two-stage application of the capillary effect, thereby further improving the injection of a substance into an organic skin.

### BRIEF DESCRIPTION OF THE DRAWINGS

FIG. 1 is a schematic drawing of a piercing element with a capillary injection needle.

FIGS. 2a, 2b, 2c and 2d are schematic drawings of piercing tips created by direct machining the end of a capillary tube in order to form a capillary injection needle.

FIGS. 3a, 3b, 3c and 3d are schematic drawings of piercing tips that are produced separately and affixed to or in capillary tubes in a separate production step to form a capillary injection needle.

FIGS. 4a and 4b are schematic drawings of piercing elements with multiple capillary injection needles.

### DETAILED DESCRIPTION

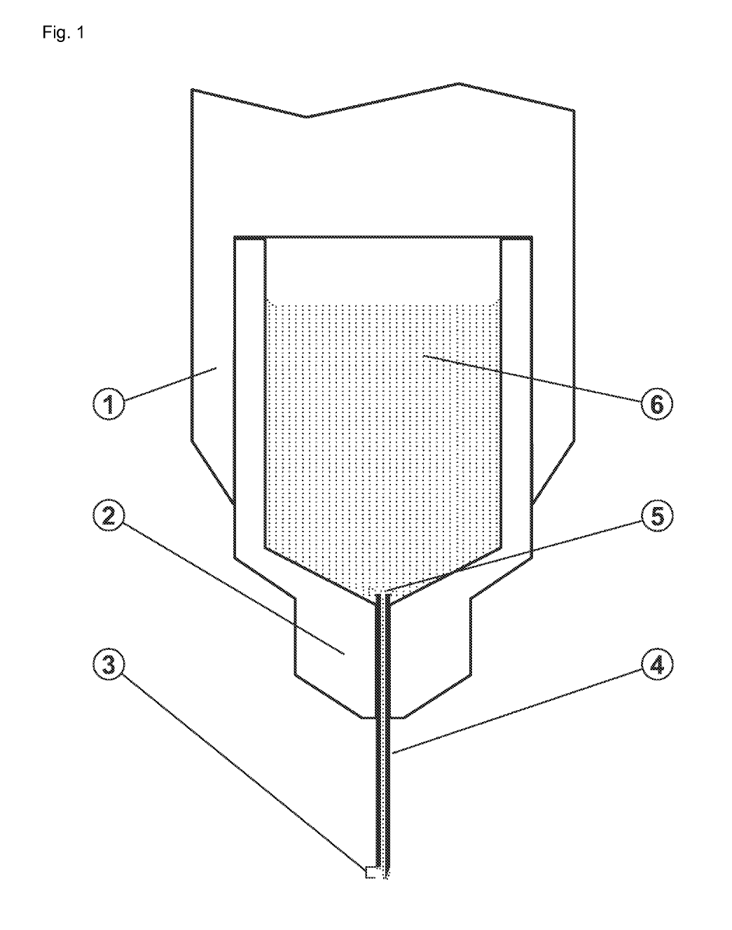

FIG. 1 shows an apparatus for the repeated injection of substances into an organic skin. A capillary injection needle 4 is rigidly connected at its proximal end with a housing 2. At an opposite distal end (treatment end) of the capillary injection needle 4 a piercing tip 3 is formed by directly machining a capillary tube. The housing 2, along with the capillary injection needle 4 fixedly installed therein, is coupled removably to a drive 1. The drive 1 provides a repetitive lifting motion to pierce the piercing tip 3 into an organic skin. Assisted by the capillary effect of the capillary injection needle 4, a substance 6 enters into the capillary tube through the proximal end 5 of the capillary injection needle 4. With each piercing of the skin by the piercing tip 3, a small quantity of the substance 6 is left behind in the skin. If no substance 6 is used, the piercing into the skin occurs analogously to the description above, but the skin is merely injured in a defined manner, which stimulates its regeneration.

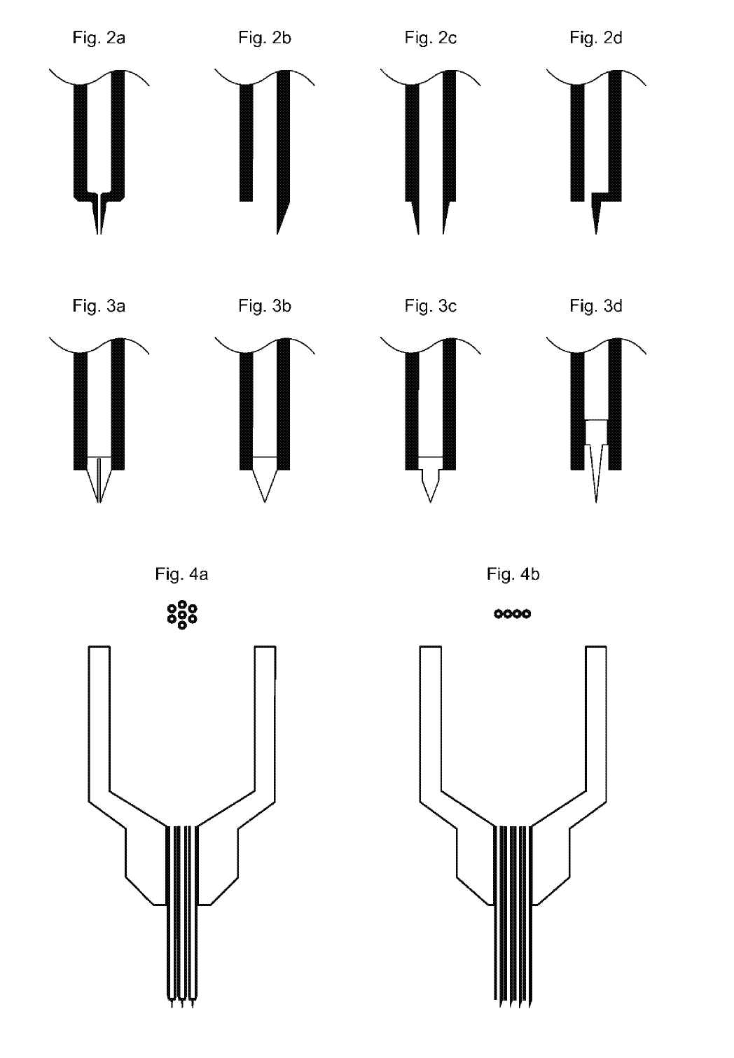

FIGS. 2a and 3a each show an example of a particularly advantageous capillary injection needles utilizing a two-stage capillary effect. The substance is transported to the piercing tip by the capillary effect of the capillary tube, and then transported to its tip through the further capillary effect of the divided piercing tip. The capillary slit of the piercing tip is thus always moistened up to its forward end with the substance, and the substance can penetrate into a skin to the full piercing depth without much of the substance being stripped during injection.

FIGS. 2a through 2d illustrate by way of example various forms of tips produced directly by working the end of the capillary tube. FIGS. 2b and 2d, for example, are optimized to prevent clogging of the capillary injection needle with skin remnants. FIGS. 2a and 2d guide the piercing tip into the center of the capillaries so that they are optimally moistened with the substance. FIG. 2c shows a double tip which can inject a large quantity of substance into an organic skin per stroke. The shown embodiment provides unhindered and definable transporting of the substance through the capillary and the possibility of forming advantageous tip shapes of the piercing tip.

FIGS. 3a through 3d show by way of example various different tip forms which are first manufactured separately before being affixed to or in the capillary tube in a subsequent step in order to form a capillary injection needle. This embodiment allows creating centered tip forms of the piercing tip which could not be produced as an integral part of a capillary tube.

FIG. 4a and FIG. 4b show by way of example arrangements of piercing elements made from multiple capillary injection needles. There is essentially no limit to the number of capillary injection needles which may be used, nor to variety of configurations in which they may be arranged.

While the present invention has been described with reference to exemplary embodiments, it will be readily apparent to those skilled in the art that the invention is not limited to the disclosed or illustrated embodiments but, on the contrary, is intended to cover numerous other modifications, substitutions, variations and broad equivalent arrangements that are included within the spirit and scope of the following claims.

What is claimed is:

1. An apparatus for repeating injection of a substance into an organic skin, comprising:
a housing configured to hold a substance;
a drive for effecting a reciprocating movement of the housing;
a capillary injection needle having a proximal end rigidly connected to the housing; and
a piercing tip arranged at a distal end of the capillary injection needle,
wherein the capillary injection needle is configured to transport the substance by capillary force from the proximal end at the housing to the piercing tip the distal end and moisten the piercing tip with the substance.

2. The apparatus as in claim 1, wherein the capillary injection needle is hollow, and provides for unobstructed flow of the substance through the capillary injection needle.

3. The apparatus as in claim 1, wherein the piercing tip of the capillary injection needle is integrally formed on the distal end of the capillary injection needle.

4. The apparatus as in claim 1, wherein the piercing tip of the capillary injection needle is a separately formed component which is affixed to a capillary tube to form the capillary injection needle.

5. The apparatus as in claim 1, comprising more than one capillary injection needle.

6. A method for injecting a substance into an organic skin, comprising:
providing the apparatus as in claim 1;
filling the housing with the substance; and
penetrating the organic skin with the capillary injection needle.
